(19) United States
(12) Patent Application Publication
(10) Pub. No.: US 2013/0324500 A1
(43) Pub. Date: Dec. 5, 2013

(54) SPINAL CORD INJURY, INFLAMMATION AND IMMUNE-DISEASE: LOCAL CONTROLLED RELEASE OF THERAPEUTIC AGENTS

(71) Applicant: InVivo Therapeutics Corporation, Cambridge, MA (US)

(72) Inventors: Christopher D. Pritchard, Cambridge, MA (US); Robert S. Langer, Newton, MA (US); Francis M. Reynolds, Lafayette Hill, PA (US); Eric J. Woodard, Jamaica Plain, MA (US)

(73) Assignee: InVivo Therapeutics Corporation, Cambridge, MA (US)

(21) Appl. No.: 13/962,459

(22) Filed: Aug. 8, 2013

Related U.S. Application Data
(63) Continuation of application No. 12/567,589, filed on Sep. 25, 2009.
(60) Provisional application No. 61/100,127, filed on Sep. 25, 2008.

(51) Int. Cl. A61K 9/00; A61K 45/06; A61K 31/65; A61K 31/573

(52) U.S. Cl. CPC A61K 9/0019; A61K 31/573; A61K 45/06; A61K 31/65
USPC 514/154; 514/179; 514/171

(57) ABSTRACT

A drug delivery system is provided for treatment of oxidative stress. The drug delivery system can include a therapeutic agent and a matrix. The therapeutic agent can include an antioxidant or steroid. The matrix can include a hydrogel, particle, microparticle, or nanoparticle. A method of treating injury, including peripheral nerve injury or spinal cord injury, is also provided. The method includes injecting the drug delivery system at the site of injury.

This application is a continuation of U.S. patent application Ser. No. 12/567,589, filed Sep. 25, 2009, which claims the benefit of U.S. provisional patent application No. 61/100,127, which was filed on Sep. 25, 2008, both of which are incorporated herein by reference as if fully set forth.

### FIELD OF INVENTION

The disclosure herein relates to therapeutic agents delivered to the site of injury.

### BACKGROUND

Nitric Oxide (NO) is a gaseous chemical messenger, involved in a variety of physiological processes throughout the human body. It is found in highest concentrations in the central nervous system (CNS). NO synthesis is catalyzed by the enzyme NO synthase (NOS) (Conti, A., Miscusi, M., Cardali, S., Germano, A., Suzuki, H., Cuzzocrea, S., and Tomasello, F. (2007) Nitric oxide in the injured spinal cord: Synthases cross-talk, oxidative stress and inflammation. Brain Research Reviews 54, 205-218). There are four isoforms of NOS in the CNS. Two are expressed constitutively: neuronal (nNOS) and endothelial (eNOS). A functionally active isoform is found in mitochondria (mtNOS), and the fourth is inducible under pathological conditions (iNOS).

Under normal conditions, nNOS is localized in neurons, perivascular nerves, and at very low levels in astrocytes. eNOS can be found in cerebrovascular endothelium. iNOS is expressed in astrocytes, microglia, vascular smooth muscle and endothelial cells.

In addition to its role during normal function, however, NO can have toxic affects. NO can outcompete superoxide dismutase for superoxide anion radical (O₂^{-}), forming peroxynitrite anion. Peroxyintrite itself can be toxic. In addition, under physiological conditions, peroxynitrite decomposes into hydroxyl radical, carbonate radical, and nitrogen dioxide, all of which subject cells to toxic oxidative stress.

Oxidative stress due to peroxynitrite and its decomposition products is implicated in a plethora of disease and injury states, including spinal cord injury (SCI), stroke, myocardial infarction, chronic heart failure, diabetes, circulatory shock, chronic inflammatory diseases, cancer, and neurodegenerative disorders.

Following neuronal injury, nNOS is up-regulated for a short time period (1 hour). Evidence suggests this contributes to ischemic damage. On the other hand, eNOS produced NO may play a neuroprotective role by promoting vasodilatation and inhibiting micro-vascular aggregation and adhesion. It is hypothesized that NO in this context may have a protective function, scavenging reactive oxygen species (ROS) produced during ischemia. However, following the initial up-regulation of nNOS, down-regulation of NO below constitutive levels may contribute to oxidative stress and the hyper-induction of iNOS.

iNOS is expressed in virtually all cell types under pathological conditions such as inflammation, immune response and trauma. Induction requires inflammatory cytokines, leading to activation of transcription factors STAT-1 and (NF)-κB. Once expressed, iNOS produces spatiotemporally highly concentrated NO. Although important in the phagocytic process, excess NO may cause damage to tissues when released in an uncontrolled manner, as observed during chronic inflammation, auto-immune disease, and trauma.

In SCI, iNOS mRNA is expressed in damaged tissue just 2 hours after injury and continues for several days. Inflammatory cells do not invade tissue prior to 3 hours post-injury. Therefore, early iNOS expression following SCI is likely due only to resident spinal cord cells, in particular microglia. iNOS expression after this time point is mainly due to infiltrated inflammatory cells. Neutrophils can be detected in the spinal cord 1 hour after injury, but are mainly intravascular. Extravasation occurs 3 to 4 hours post-injury. Neutrophil prevalence reaches a maximum 1 to 3 days post-injury and is elevated for up to 10 days. Neutrophils release a number of substances including chemokines, cytokines, enzymes, ROS and reactive nitrogen radicals.

NO is involved in neurotoxicity after ischemic and traumatic injuries in the CNS (Xiong, Y, Rabchevsky, A. G. and Hall, E. D. (2007) Role of peroxynitrite in secondary oxidative damage after spinal cord injury. J. Neurochem. 100 (1). 639-649). NO as a free-radical can cause protein nitrosylation. It can attenuate oxidative phosphorylation and inhibit glycosylation via a number of mechanisms, resulting in energy depletion, oxygen starvation, and neuronal death. NO can promote mutagenic DNA deamination and cause phospholipid peroxidation, damaging the structural and functional integrity of cell membranes and leading to cell death.

After SCI, studies indicate sustained elevated levels of peroxynitrite formation for at least one week post-injury, which coincides with protein oxidation and lipid peroxidation. See Deng, Y., Thompson, B. M., Gao, X. and Hall, E. D. (2007) Temporal relationship of peroxynitrite-induced oxidative damage, calpain-mediated cytoskeletal degradation and neurodegeneration after traumatic brain injury. Exp. Neurol. 205. 154-165. Further studies indicate efficacy of a number of agents in mitigating secondary injury, including penicillamine, tempol (Hillard, V. H., Peng, H., Zhang, Y., Das, K., Murali, R. and Etlinger, J. D. (2004) Tempol, a nitroxide antioxidant, improves locomotor and histological outcomes after spinal cord contusion in rats, J Neurotrauma 21 (10). 1405-1414 (“Hillard et al.”)), and uric acid (Scott, G. S., Cuzzocrea, S., Genovese, T., Koprowski, H., and Hooper, D. C. (2005) Uric acid protects against secondary damage after spinal cord injury. Proc. Natl. Acad. Sci. 102 (9), 3483-3488 (“Scott et al.”)). Additionally, studies suggest a neuroprotective effect of clinically administrated glucocorticoid steroids is in large part due to inhibition of lipid peroxidation, rather than receptor mediated anti-inflammation (Hall, E. D. and Springer, J. E. (2004) Neuroprotection and Acute Spinal Cord Injury: A Reappraisal. NeuroRx. 1, 80-100).

In 1990, high dose Methylprednisolone was adopted as the Standard of Care for acute SCI. The administration of steroids for acute SCI is, however, controversial primarily due to risks of adverse side effects (e.g., infection, pneumonia, septic shock, diabetic complications, and delayed wound healing) and dosage difficulties (e.g., a sharp biphasic dose-response curve and variations over the required treatment duration depending upon the initiation time-point). Methylprednisolone has been administered locally. See Chvatal, S. A., Kim, Y.-T., Bratt-Leal, A. M., Lee, H., and Bellamkonda, R. V. (2008) Spatial distribution and anti-inflammatory effects of Methylprednisolone after sustained local delivery to the contused spinal cord. Biomaterials, 1-9. The administration in Chvatal et al. required surgical exposure of the spinal cord through laminectomy. Further, the delivery medium, warm agarose, was applied outside of the dura.

Oxidative stress to spinal cord cells post SCI and peripheral nerves post injury can be attributed to NO and ROS formed peroxynitrite and peroxynitrite reactive decomposition products under physiological conditions. The necrotic processes or apoptotic cascades resulting from oxidative stress due to peroxynitrite is characteristic of lesion expansion following spinal cord injury or injury to peripheral nerves.

### SUMMARY

In an aspect, the invention relates to a method of treating injury at a site of the injury in a patient. The method includes administering a drug delivery system having a matrix and one or more therapeutic agents to the patient at the site of injury.

In another aspect, the invention relates to a drug delivery system having a matrix and one or more therapeutic agents.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 shows an 1H-NMR spectrum for PEG-4000 (Fluka).

FIG. 2 shows an 1H-NMR spectrum for PLGA 50:50 Lactel.

FIG. 3 shows an 1H-NMR spectrum for CP-PLGA-pPEG-PLGA-1.

FIG. 4 shows an 1H-NMR spectrum used to detect the chain transfer agent CP-PLGA-pPEG-PLGA-RAFT-funct.

FIG. 5 shows an ¹H-NMR spectrum of S-(thiobenzoyl) thioglycolic acid chloride DJS-CP-thiobenzoyl-thioglycolic acid chloride-1.

FIG. 5a shows an expanded portion of the 1H-NMR spectrum illustrated in FIG. 5.

FIG. 6 shows an 1H-NMR spectrum for CP-PLGA-PEG-PLGA-CTA-Cl-rxn-1.

FIG. 7 shows an ¹H-NMR spectrum for CP-PGS-CTA poly(glycerol-co-sebacic acid) functionalized with a S-thiobenzoyl-thioglycolic acid chain transfer agent.

FIG. 8 shows a therapeutic agent release curve from hydrogel-microparticles with 5 mg microparticles in 50 μL hydrogel.

### DETAILED DESCRIPTION OF THE PREFERRED EMBODIMENTS

The words “a,” and “one,” as used in the claims and in the corresponding portions of the specification, are defined as including one or more of the referenced item unless specifically stated otherwise.

As used herein, “matrix” refers to a hydrogel, particle, nanoparticle, microparticle, or combinations thereof.

As used herein, “therapeutic agent” and “drug” are used interchangeably.

As used herein, “injury” refers to injury caused by any means including but not limited to physical trauma, disease, immune disease, or inflammation.

As used herein, “patient” refers to a human or non-human animal within the phylum chordata.

As used herein, “pharmaceutically acceptable salt” or “pharmaceutically acceptable salts” means those salts of compounds that are safe and effective for use in a patient and that possess the desired biological activity. Pharmaceutically acceptable salts include salts of acidic or basic groups. Pharmaceutically acceptable acid addition salts include but are not limited to hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, sodium succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Pharmaceutically acceptable salts include salts with various amino acids. Pharmaceutically acceptable base salts include but are not limited to aluminum, calcium, lithium, magnesium, potassium, sodium, zinc, and diethanolamine salts.

The embodiments herein provide a strategy for modulating post-traumatic secondary injury by scavenging radicals in the injured site or site of inflammation through local administration of therapeutic agents. Due to undesirable side-effects accompanying systemic administration of drugs (e.g., glucocorticoidal steroids), local administration of therapeutic agents, including anti-inflammatory drugs (e.g., minocycline or methylprednisolone), or free-radical scavengers (e.g., uric acid or tempol), to mitigate secondary injury could be significant. To affect local administration of a therapeutic agent, a drug delivery system targeting processes responsible for nerve damage following injury is provided. The processes targeted include oxidative stress resulting from damage caused by injury. Embodiments of the drug delivery system can be adapted for treatment of the spinal cord after SCI. However, embodiments of the drug delivery system may be used at any site of injury or inflammation. The site of injury or inflammation may be the spinal cord or peripheral nerves. Methods of treatment with the drug delivery system may be directed to intra-cellular regions, extra-cellular regions, intravascular regions and/or cell membranes. Embodiments of the drug delivery system and methods can address deleterious effects of inflammation and these embodiments can be used for the treatment of chronic inflammation, auto-immune disease, spinal cord injury (SCI), stroke, myocardial infarction, chronic heart failure, diabetes, circulatory shock, chronic inflammatory diseases, cancer, neurodegenerative disorders, traumatic brain injury, severing of peripheral nerves, nerve root impingement, and other disorders or traumatic injuries. The drug delivery system is a device that includes a matrix and one or more therapeutic agents. The methods of treatment include administering the drug delivery system.

The drug delivery system can include but is not limited to the following combinations of matrix and therapeutic agent: 1) hydrogel plus therapeutic agent; 2) hydrogel plus a combination of multiple therapeutic agents; 3) particles and therapeutic agent; 4) particles plus multiple therapeutic agents; 5) hydrogel plus particles plus therapeutic agent, where the agent is located in the hydrogel, particles or both; 6) hydrogel plus particles plus multiple therapeutic agents, where the therapeutic agents are localized in the hydrogel, the particles (perhaps a distinct set of particles) within the hydrogel, or both; and 7) hydrogel plus particles plus multiple therapeutic agents, where particular therapeutic agents are localized in the hydrogel, the particles (perhaps a distinct set of particles) within the hydrogel, or both. The particles can be microparticles or nanoparticles. The therapeutic agent or therapeutic agents can be dissolved or dispersed in the hydrogel, the particles, or the hydrogel and particles for controlled release kinetics via diffusion and/or dissolution. Preferably, the matrix is injectable and the drug delivery system can be delivered to a position at the site of injury through injection. The drug delivery system may, however, be administered by other methods, which include but are not limited to surgical implantation of the drug delivery device.

The hydrogel can be a temperature-sensitive biodegradable composition. As used herein, “temperature sensitive” means that the hydrogel exhibits a sol-gel phase transition between temperatures below patient body temperature and the patient body temperature, or that mixtures of polymers react to form a hydrogel at temperatures closer to patient body temperature more readily than at lower temperatures. Preferably, the patient is human, the body temperature is 37°, and the lower temperature can be room temperature (e.g., 22° C.). Temperature-sensitive hydrogels may have a critical temperature closer to the body temperature of the patient, preferably closer to 37° C.

A biodegradable, temperature-sensitive hydrogel in the drug delivery system can include multi-block co-polymers. One or more of the polymer blocks can be biodegradable, biocompatible, or biodegradable and biocompatible. Some of the polymer blocks can be biodegradable while others are biocompatible. Preferably, the hydrogel components, for example polymers, monomers or breakdown products, are biodegradable; biocompatible; or biocompatible and excretable. The hydrogel may include biocompatible polymer blocks that can be excreted by the body.

Ester links between monomer blocks are hydrolysable and can be degraded in vivo to release polymer monomer blocks. Polymers containing ester bonds are biodegradable. Amide, anhydride, and ether links may also be hydrolysable. These links and others that can be degraded by action of enzymes, reducing conditions (e.g., thioester, thioether, disulfide links) or conditions present within the patient may also be used in the polymers contemplated for a hydrogel or particle. The hydrogel may contain polymers blocks having ethylene glycol with ether links, oligoethylene glycol, or polyethylene glycol, which are biocompatible and can be excreted. Polymers of glycolic acid, lactic acid, glycerol, and sebacic acid are biodegradable hydrogels may include these polymers. Lactide, glycolide, poly(glycerol-co-sebacic acid) polymers are biodegradable and one or more of these polymers can be included in the hydrogel. Polymers contemplated as part of the hydrogel include but are not limited to poly(glycerol-co-sebacic acid) acrylate; multiblock copolymers of poly(lactide-co-glycolide) and poly(ethylene glycol) or oligo (ethylene glycol) methyl methacrylate; and graft copolymers of poly(glycerol-co-sebacic acid) and poly(ethylene glycol), oligo (ethylene glycol) methyl methacrylate or poly(N-isopropylacrylamide), ethoxylated trimethylolpropane tri-3-mercaptopropionate, or poly(ethylene glycol)diacrylate. These polymers are temperature-sensitive.

Hydrogels can swell or shrink under changing physical conditions, which are of physiologic significance. For example changes in temperature, pH or ionic strength can cause a hydrogel to swell or shrink. Preferably, a hydrogel injected into the spinal cord or other site of injury does not swell significantly upon equilibrating to conditions within the injury site. A swelling hydrogel may, however, be included in the drug delivery system.

Hydrophilic polymers containing ethylene glycol monomer units with reactive end groups, which include but are not limited to acrylate, methacrylate, vinyl, dihydrazide or thiol groups, can be used as polymers in either a hydrogel or particle. Acrylochloride, methacrylochloride, vinyl chloride can be used to form the acrylate or methacrylate end groups. Thiol end groups can be provided by mercaptopropionic acid, cysteine, and cystamine. Multiple synthesis methods, such as ring-opening-polymerization and living radical polymerization may be employed to produce polymers for the matrix. The hydrogel may be composed of a covalently or physically cross-linked network. Physical cross links refer to the aggregation of hydrophic blocks.

Polymers having acrylate, methacrylate, vinyl, dihydrazide or thiol functionalized compounds are capable of reacting with other polymers having compatible reactive end groups to form hydrogels. Acrylate, methacrylate and vinyl reactive end groups are all compatible with each other and with thiols. Thiol and acrylate functionalized polymers or polymer blocks are capable of reacting to form thiol-ethers under mild conditions (heat or light). Therefore, thiol and acrylate functionalized water soluble polymers are suitable candidates for hydrogels in the drug delivery system. Some hydrogels can swell or shrink upon equilibration following gelation, which may be due to incomplete conversion or to the high concentration of reactants required for a sufficiently rapid reaction compared to subsequent equilibrium concentrations in the gel under physiological conditions (e.g., temperature, pH, ionic strength). The thiol-acrylate functionalized polymers are attractive polymers in drug delivery devices, due to rapid reaction rates and high extents of conversion to hydrogel. A hydrogel for the drug delivery system may be formed by mixing polymers with compatible reactive end groups. Preferably, the mixture includes a thiol containing polymer and an acrylate containing polymer. After mixing, the formation of thiol-esters forms the hydrogel when the combination is exposed to sufficient temperatures or light. Preferably, thiol-ester formation occurs more rapidly at body temperature of the patient then at temperatures lower than the body temperature of the patient. For example, the thiol-ester formation may proceed more rapidly at or near 37° C. than at or near 22° C. The polymers with compatible reactive end groups, preferably thiol containing and acrylate containing polymers, can be mixed prior to or during administration. When mixed during administration, the polymers can be mixed while being implanted, or mixed by serial or parallel injections of the different polymers. A non-limiting example of such a mixture includes ethoxylated trimethylolpropane tri-3-mercaptopropionate in combination with poly(ethylene glycol)diacrylate.

A hydrogel in the drug delivery system preferably has a compressive modulus similar to that of tissue surrounding the injury site. For example, a drug delivery system designed to be delivered to the spinal cord can have a compressive modulus similar to that of the spinal cord. The porosity of a hydrogel in the drug delivery system can be matched to the size of the therapeutic agent to be released. If a 500 dalton therapeutic agent is part of the drug delivery system, the mesh of the hydrogel should allow a 500 dalton therapeutic agent to migrate through the gel.

The matrix may include particles containing drug and the particles may provide for controlled release kinetics of the drug. In a preferred embodiment, the particles can be injected as a suspension into the area of damage, which may be at a peripheral nerve or the spinal cord. The particles can be microparticles, ranging in size from about 1 micron to about 1000 microns. The particles can be nanoparticles, ranging in size from about 1 nanometer to about 1000 nanometers. The particle dimensions can, however, vary to suit the particular application. The therapeutic agent can be present on or within the particle at a concentration effective to achieve the effect of scavenging radicals, preventing the formation of radicals, or otherwise counteracting the toxic effects of nitric oxide associated oxidative stress. In preferred embodiments, the particle contains therapeutic agent at 0.1-30%, more preferably 1-30% w/w ((weight of drug)/(weight of particle plus drug)). The therapeutic agent can be released by mechanisms of diffusion, dissolution, and particle degradation.

The particle may be a solid polymer or a gel. In a preferred embodiment, particles are made of a biodegradable, biocompatible polymer, which can be, for example, a polyester. One suitable polyester for a particle is poly(lactide-co-glycolide) (PLGA), which degrades by ester hydrolysis. Other suitable materials include polylactide, polyglycolide, and poly(carboxyphenoxy propane)-co-sebacic acid) (e.g., gliadel Wafer™ from MGI Pharmaceuticals). Preferably, the particle components, for example polymers, monomers or breakdown products, are biodegradable; biocompatible; or biocompatible and excretable.

A combination of hydrogel and particles can be provided to the combination to provide the ability to decouple release kinetics from in-situ gelling. Through decoupling of release kinetics, the release of therapeutic agent from particles versus hydrogel may occur at different rates. In a preferred embodiment, different therapeutic agents may be included in the hydrogel or particles, or different types of particles, such that each particular therapeutic agent is released at a different rate.

In an embodiment, hydrogel or particle material is functionalized with a therapeutic agent. The therapeutic agent may be attached to by any type of bond to functionalize the particle. Preferably, the attachment is through carboxyl or hydroxyl groups of polymer repeating units through ester, amide, ether, or acetal bonds. In a preferred embodiment, a functionalized hydrogel includes therapeutic agent attached to a poly(glycerol-co-sebacate)acrylate (PGSA).

Any therapeutic agent can be used to functionalize a hydrogel or particles, but in a preferred embodiment the therapeutic agent is attached to a hydrogel and the therapeutic agent is an antioxidant. More preferably, the antioxidants ascorbic acid (vitamin C) and alpha-tocopherol (vitamin E) are attached to the hydrogel. Vitamin C and vitamin E antioxidants when present in combination can recycle each other and antioxidant properties can be extended. Other therapeutic combinations can be employed to effect recycling in the drug delivery system.

PGSA can form elastomeric networks under mild conditions (radical polymerization), which can protect the antioxidant from denaturing during processing. In addition, a scaffold of arbitrary geometry can be formed from PGSA using melt molding or solid free form rapid prototyping techniques. This can be used to customize the drug delivery system to a particular lesion cavity (or spinal cord tumor cavity), resulting in an improvement of surgical intervention to treat injury, which may be SCI or peripheral nerve injury.

Table I, below, lists an exemplary formulation for the following seven drug delivery system combinations: 1) hydrogel plus therapeutic agent; 2) hydrogel plus a combination of multiple therapeutic agents; 3) particles and therapeutic agent; 4) particles plus multiple therapeutic agents; 5) hydrogel plus particles plus therapeutic agent, where the agent is located in the hydrogel, particles or both; 6) hydrogel plus particles plus multiple therapeutic agents, where the therapeutic agents are localized in the hydrogel, the particles (perhaps a distinct set of particles) within the hydrogel, or both; and 7) hydrogel plus particles plus multiple therapeutic agents, where particular therapeutic agents are localized in the hydrogel, the particles (perhaps a distinct set of particles) within the hydrogel, or both.

TABLE I

| | Hydrogel | Particle(s) | Therapeutic agent(s) | Other constituents |
|---|---|---|---|---|
| Combination 1 | 25 wt. % ETTMP1300, | | Methylprednisolone sodium | PBS, pH 7.4 |
| | 25 wt. % PEGDA400 | | succinate, 0.1 wt. % | water |
| Combination 2 | 25 wt. % ETTMP1300, | | Methylprednisolone sodium | PBS, pH 7.4 |
| | 25 wt. % PEGDA400 | | succinate, 0.1 wt. % | water |
| | | | Minocycline, 0.1 wt. % | |
| Combination 3 | | PLGA microparticles, 1-100 | Methylprednisolone sodium | |
| | | microns, 10 wt. % | succinate, 0.1 wt. % | |
| Combination 4 | | PLGA microparticles, 1-100 | Methylprednisolone sodium | |
| | | microns, 10 wt. % | succinate, 0.1 wt. % | |
| | | | Minocycline, 0.1 wt. % | |
| Combination 5 | 25 wt. % ETTMP1300, | PLGA microparticles, 1-100 | Methylprednisolone sodium | PBS, pH 7.4 |
| | 25 wt. % PEGDA400 | microns, 10 wt. % | succinate, 0.1 wt. % | water |
| Combination 6 | 25 wt. % ETTMP1300, | PLGA microparticles, 1-100 | Methylprednisolone sodium | PBS, pH 7.4 |
| | 25 wt. % PEGDA400 | microns, 10 wt. % | succinate, 0.1 wt. % | water |
| | | | Minocycline, 0.1 wt. % | |
| Combination 7 | 25 wt. % ETTMP1300, | PLGA microparticles, 1-100 | Methylprednisolone sodium | PBS, pH 7.4 |
| | 25 wt. % PEGDA400 | microns, 10 wt. % | succinate, 0.1 wt. % | water |
| | | | Minocycline, 0.1 wt. % | |

In the examples of Table I, wt % is calculated as the weight of the constituent divided by the weight of the combination, which includes the other constituents. The PBS, pH 7.4 is phosphate buffered saline at pH 7.4, which includes 144 mg/L (1.06 mM) potassium phosphate monobasic (KH₂PO₄, 136 g/mol), 9000 mg/L (155.17 mM) sodium chloride (NaCl, 58 g/mol), and 795 mg/L (2.97 mM) sodium phosphate dibasic (Na₂HPO₄-7H₂O).

Any molecule acting as a radical scavenger or anti-inflammatory agent is a candidate therapeutic agent for the drug delivery system. Preferably, the molecule is a small molecule. Therapeutic agent(s) can preferably reduce the number of free-radicals and/or reduce the production of free radicals at a locale within the body. The drug delivery system can include combinations of more than one therapeutic agent. The therapeutic agent can be an antioxidant, a steroid, or combinations thereof. In a preferred embodiment, the therapeutic agent includes one or more substance selected from the group of an antioxidant or antioxidants, tempol (4-hydroxy-2,2,6,6-tetramethylpiperidine-1-oxyl), uric acid, minocycline, methylprednisolone, MnTBAP (Manganese (III) tetrakis(4-benzoic acid)porphyrin), and dexamethasone. The antioxidant may be, but is not limited to ascorbic acid or alpha-tocopherol. Combinations of therapeutic agents that can recycle each other may also be provided in the drug delivery system. For example, ascorbic acid (vitamin C) and alpha-tocopherol (vitamin E) can be used in combination to recycle each other and extend antioxidant properties.

Therapeutic agent(s) are not limited to those above. Non-limiting examples of therapeutic agents that could be included in the drug delivery system include inhibitors of NOS or NO production, antioxidants, spin traps, and peroxynitrite scavengers.

A non-limiting list of inhibitors of NOS or NO production that can be provided in the drug delivery system includes 1400 W (N-(3-(aminomethyl)benzyl)acetamidine); actinomycin D; AET; ALLM; ALLN; N^{G}-allyl-L-arginine; aminoguanidine, hemisulfate; 1-amino-2-hydroxyguanidine; p-toluenesulfonate; 2-amino-4-methylpyridine; AMITU; AMT; S-benzylisothiourea; bromocriptine mesylate; L-canavanine sulfate; canavalia ensiformis; chlorpromazine, hydrochloride; curcumin; curcuma longa L; cycloheximide; high purity cycloheximide; cyclosporine; dexamethasone, 2,4-diamino-6-hydroxypyrimidine; N^{G},N^{G}-dimethyl-L-arginine; N^{G},N^{G1}-dimethyl-L-arginine; diphenyleneiodonium; DMHP, S(−)-epigallocatechin gallate; S-ethyl-N-phenylisothiourea; 2-ethyl-2-thiopseudourea; ETPI; basic fibroblast growth factor; bovine basic fibroblast growth factor; human recombinant basic fibroblast growth factor, GED; haloperidol; L-N⁶-(1-lminoethyl)lysine, dihydrochloride; L-N⁵-(1-lminoethyl)ornithine; LY83583; LY231617; MEG; melatonin; S-methylisothiourea sulfate; S-methyl-L-thiodtrulline, dihydrochloride; N^{G}-monoethyl-L-arginine; N^{G}-monomethyl-D-arginine monoacetate; DiHABS (di-hydroxyazobenzene-p′-sulfonate) salt of N^{G}-monomethyl-L-argninine; N^{G}-monomethyl-L-argninine; monohydrate HABS salt of N^{G}-monomethyl-L-argninine; N^{G}-monomethyl-L-homoarginine; mycophenolic acid, L-NIL; inducible nitric oxide synthase inhibitor set (Calbiochem®); neuronal nitric oxide synthase inhibitor set (Calbiochem®); N^{G}-nitro-D-arginine; N^{G}-nitro-L-arginine; N^{G}-nitro-D-arginine methyl ester; N^{G}-nitro-L-arginine methyl ester; p-nitrolue tetrazolium chloride; 7-nitroindazole; sodium salt of 7-nitroindazole; 3-bromo-7-nitroindazol; sodium salt of 3-bromo-7-Nitroindazol; NOS inhibitor set (Calbiochem®), 1,3-PBITU; pentamidine isethionate; PPM-18; N^{G}-propyl-L-arginine; 1-pyrrolidinecarbodithioic acid; SKF-525A; SKF-96365; sodium salicylate; spermidine; trihydrochloride spermidine; spermine; spermine tetrahydrochloride; L-thiocitrulline; N^{α}-tosyl-Lys chloromethyl ketone; N^{α}-tosyl-Phe chloromethyl ketone; TRIM; and zinc (II) Protoporphyrin IX. Pharmaceutically acceptable salts of an inhibitor or inhibitors of NOS or NO production can be included in the drug deliver system.

A non-limiting list of antioxidants that can be in the drug delivery system includes N-acetyl-L-cysteine; N-acetyl-5-farnesyl-L-cysteine; AG 1714; ambroxol hydrochloride; antioxidant set (Calbiochem®); L-ascorbic acid; bilirubin, bilirubin free acid, caffeic acid, CAPE; carnsol; (+)-catechin; ceruloplasmin; human plasma ceruloplasmin; coelenterazine; copper diisopropylsalicylate; deferoxamine mesylate; R-(−) -deprenyl hydrochloride; DMNQ; DTPA; dianhydride; ebselen; ellagic acid; dehydrate ellagic acid; (−)-epigallocatechin gallate; L-ergothioneine; dihydrate EUK-8; apo-ferritin, equine spleen apo-ferritin; cadmium free ferritin; equine spleen cadmium free ferritin; human liver ferritin; human recombinant ferritin H-chain; human recombinant ferritin L-chain; formononetin; reduced glutathione; reduced glutathione free acid; gluthathione monoethyl ester; α-lipoic acid; dihydro-DL-α-lipoic acid; luteolin, LY 231617; penicillamine; MCI-186; MnTMPyP, morin hydrate; NCO-700; NDGA; p-nitroblue tetrazolium chloride; 0-trensox; propyl gallate; resveratrol; rosmarinic acid; (+)-rutin hydrate; silymarin group; L-stepholidine; stephania intermedica; (±)-taxifolin; tetrandrine; DL-thioctic acid; thioredoxin; human recombinant low endotoxin thioredoxon; thioredoxin II; yeast thioredoxin II; recombinant yeast thioredoxin II; DL-α-tocopherol; tocopherol set (Calbiochem®); DL-α-tocopherol acetate, tocotrienol set (Calbiochem®), Trolox®; U-74389G; U-83836E; uric acid; and vitamin E succinate. Pharmaceutically acceptable salts of an antioxidant or antioxidants can be included in the drug deliver system.

Spin trap agents that can be in the drug delivery system include but are not limited to N-tert-butyl-α-phenylnitrone, tempol, and DTCS (Iron (II) N-(dithiocarboxy)sarcosine Fe2+). Pharmaceutically acceptable salts of a spin trap or spin traps can be included in the drug deliver system.

Peroxynitrite Scavengers that can be in the drug delivery system include but are not limited to ebselen; FeTMPyP; FeTPPS; reduced glutathione; reduced glutiathione free acid; melatonin; MnTBAP; MnTMPyP; L-selenomethionine; and Trolox®. Pharmaceutically acceptable salts of a peroxynitrite scavenger or peroxynitrite scavengers can be included in the drug deliver system.

Therapeutic agent(s) can be provided at any concentration effective to scavenge radicals, prevent formation of radicals, or otherwise counteract the toxic effects of nitric oxide associated stress. Preferably, therapeutic agent(s) are present in the drug delivery system at a concentration of 0.1-30% w/v (weight of drug/volume of drug delivery system). The concentration of selected therapeutic agents in a drug delivery device is provided in Table II, below.

TABLE II

| | Suggested Concentration | |
|---|---|---|
| Therapeutic Agent | Range | Suggested Concentration. |
| Vitamin C | 0.1-30%(w/v) | 0.1% (w/v) |
| Vitamin C and | 0.1-30% (w/v) vitamin C | 0.2% (w/v) (0.1% |
| vitamin E | and 0.1-30% (w/v) | vitamin C and 0.1% |
| | vitamin E | vitamin E) |
| Tempol | 0.1-30% (w/v) | 0.1% (w/v) |
| Uric acid | 0.1-30% (w/v) | 0.1% (w/v) |
| Minocycline | 0.1-30% (w/v) | 0.1% (w/v) |
| Methylprednisolone | 0.1-30% (w/v) | 0.1% (w/v) |
| MnTBAP | 0.1-30% (w/v) | 0.1% (w/v) |
| Dexamethasone | 0.1-30% (w/v) | 0.1% (w/v) |

The drug delivery system may include pharmaceutical additives such as carriers and the like. The term “carrier” as used herein includes acceptable adjuvants and vehicles. Pharmaceutically acceptable carriers can be selected from but are not limited to those in the following list: ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts or electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, and polyethylene glycol.

The therapeutic agent can be provided in a small volume of diluent as the carrier. For example, a one ml methylprednisolone sodium succinate (i.e., pregna-1,4-diene-3,20-dione,21-(3-carboxy-1-oxopropoxy)-11,17-dihydroxy-6-methyl-monosodium salt, (6α,11β) (molecular weight 496.53)) therapeutic agent solution could include 40 mg methylprednisolone sodium succinate; 1.6 mg monobasic sodium phosphate anhydrous; 17.46 mg dibasic sodium phosphate dried; 25 mg lactose hydrous; and 8.8 mg benzyl alcohol as preservative. When necessary, the pH of each formula can be adjusted. For example, sodium hydroxide could be added so that the pH of the reconstituted solution is within a range of 7 to 8 and the tonicities are, for the 40 mg per mL methylprednisolone sodium succinate solution, 0.50 osmolar. The conditions within a matrix may be designed to be the same, similar, or different than a diluent or solution that the therapeutic agent exists in prior to its addition to matrix. For example, a one ml volume of matrix with methylprednisolone sodium succinate could be designed to contain the same amount of methylprednisolone sodium succinate, monobasic sodium phosphate, dibasic sodium phosphate, lactose hydrous, benzyl alcohol, pH, and water as the diluent above. Other diluents may be utilized. The therapeutic agent can be provided in any pharmaceutically acceptable carrier. For example, the therapeutic agent can be provided in a buffered diluent, for example phosphate buffer or phosphate buffered saline.

The drug delivery system can be assembled by dissolving or soaking polymer in a therapeutic agent solution. When combinations of different particles or particle and hydrogel are used, the matrix components can be exposed to the therapeutic agent as a combination or separately. If different therapeutic agents are intended for particles, different subsets of particles, or hydrogels, these components can be exposed to the respective therapeutic agent solution prior to combining all of the polymer components.

Computational models of degradation, drug release and in vivo distribution have been developed to predict the effect of various design parameters on the spatial-temporal drug profile of the drug delivery systems. Parameters include polymer composition, molecular weight, polydispersity, drug type, drug size, drug-polymer interactions and geometry of the drug delivery system. The parameters can be adjusted to optimize the drug delivery system.

As illustrated in example 7, below, ethoxylated trimethylolpropane tri-3-mercaptopropionate with a MW=1300 g/mol mixed with poly(ethylene glycol)diacrylate with a MW 400 g/mol as a hydrogel and a PLGA polymer with a MW 11,600 g/mol is a non-limiting example of a hydrogel and particles with parameters suitable for an embodiment of the drug delivery system. Hyrdrogels and particles with other parameters may be utilized in a drug delivery system.

The drug delivery system can be injected into a patient at an area at the site of injury or inflammation, or deposited into or at the site following surgery to expose the area. By injection, rather than surgical intervention, the drug delivery system can be administered in a minimally invasive manner. In a preferred embodiment, only one administration would be necessary to maintain a sustained dosage. The drug can thus be delivered directly to the point of injury or inflammation, thereby minimizing side-effects related to systemic administration. Preferably, but not exclusively, a hydrogel made by a combination of ethoxylated trimethylolpropane tri-3-mercaptopropionate with poly(ethylene glycol)diacrylate may be utilized in this method. Non-limiting examples of such a drug delivery device are provided in Table I.

One method of treatment is injection of the drug delivery system into a contusion injury in the spinal cord. This can be accomplished by intradural intramedullary injection. By injecting, or otherwise implanting, the drug delivery system into the spinal cord, neither the drug nor elements of drug delivery system has to cross the dura and the drug does not have to cross-the blood brain barrier. Preferably, the drug delivery system is designed to release the therapeutic agent over a sustained period of time, synchronized with the pathophysiological increased and temporally sustained levels of free radicals and free radical production at the injury site, which is due in part to microglial activation and neutrophil infiltration. Preferably, but not exclusively, a hydrogel made by a combination of ethoxylated trimethylolpropane tri-3-mercaptopropionate with poly(ethylene glycol)diacrylate may be utilized in this method. Non-limiting examples of such a drug delivery device are provided in Table I.

In preferred embodiments, the drug delivery system is designed to degrade during treatment by hydrolysis and be excreted by the body via normal pathways, without the need for further surgical intervention. Preferably, but not exclusively, a hydrogel made by a combination of ethoxylated trimethylolpropane tri-3-mercaptopropionate with poly(ethylene glycol)diacrylate may be utilized in this method. Non-limiting examples of such a drug delivery device are provided in Table I.

Potential Drug Delivery System Tests

Therapeutic agents, matrices and combinations thereof can be tested by methods known in the art. A range of nitric oxide donors, such as SIN-1 hydrochloride, can be used in vitro to produce sustained levels of peroxynitrite and radicals due to peroxynitrite decomposition. Antioxidant activity can then be assayed by measuring nitrite using numerous methods, for example via modified Griess Reagent. A typical commercial Griess reagent contains 0.2% naphthylenediamine dihydrochloride, and 2% sulphanilamide in 5% phosphoric acid. Cell integrity in vitro can be assayed using MTT or MTS assays for mitochondrial activity of lactate dehydrogenase (LDH) for cell membrane integrity. See Mosmann, T. (1983) Rapid Colorimetric Assay for Cellular Growth and Survival: Application to Proliferation and Cytotoxicity Assays. J. Immunol. Meth. 65, 55-63; and Wilson, A. P. (2000) Cytotoxicity and Viability Assays in Animal Cell Culture: A Practical Approach, 3rd ed. (ed. Masters, J. R. W.) Oxford University Press: Oxford 2000, Vol. 1, which are incorporated by reference as if fully set forth. A drug delivery system can also be tested for its ability to reduce cell death or cell membrane damage in vitro, and also elimination of nitrites produced by donors such as SIN-1. The efficacy of a therapeutic agent, matrix, or drug delivery system can be assayed after in vivo studies via immunostaining or using markers for peroxinitrite oxidative stress. Markers include but are not limited to 3-nitrotyrosine and 4-hydroxynonenal.

The spatial and temporal characteristics of peroxynitrite-derived oxidative damage after a moderate contusion injury in rats was described in Xiong, Y, Rabchevsky, A. G. and Hall, E. D. (2007) Role of peroxynitrite in secondary oxidative damage after spinal cord injury. J. Neurochem. 100 (1), 639-649 (“Xiong et al.”), which is incorporated herein as if fully set forth. Xiong et al. showed that 3-nitrotyrosine, a specific marker for peroxynitrite, rapidly accumulated at early time points (1 and 3 h) and significantly increases in 3-nitrotyrosine were sustained out to 1 week after injury in comparison to sham rats. Additionally, Xiong et al. showed a coincident and maintained increase in the levels of protein oxidation-related protein carbonyl and lipid peroxidation-derived 4-hydroxynonenal. The peak increases of 3-nitrotyrosine and 4-hydroxynonenal were observed at 24 h post-injury. In immunohistochemical results, Xiong et al. showed the co-localization of 3-nitrotyrosine and 4-hydroxynonenal, indicating that peroxynitrite is involved in lipid peroxidative as well as protein nitrative damage. Another consequence of oxidative damage is an exacerbation of intracellular calcium overload, which activates the cysteine protease calpain leading to the degradation of several cellular targets including cytoskeletal protein (α-spectrin). Xiong et al. also showed, through analysis of α-spectrin breakdown products, that the 145-kDa fragments of α-spectrin, which are specifically generated by calpain, were significantly increased as soon as 1 h following injury although the peak increase did not occur until 72 h post-injury. Xiong et al. concluded that the later activation of calpain was most likely linked to peroxynitrite-mediated secondary oxidative impairment of calcium homeostasis. Candidate therapeutic agents, matrices and combinations may be tested for markers described in Xiong et al. Numerous methods of assaying markers may be employed, including the methods described by Xiong et al. The methods described in Xiong et al. included a rat model of traumatic spinal cord contusion, immunobolotting analysis for 3-nitrotyrosine and 4-hydroxynonenal, western blotting for α-spectrin breakdown products, and statistical analysis, as follows.

A rat model of traumatic spinal cord contusion: According to Xiong et al., all studies described therein employed young adult female Sprague-Dawley rats (Charles River, Portage, Mich., USA) weighing between 200 and 225 g. The animals were randomly cycling and were not tested for stage of the estrus cycle. They were fed and watered ad libitum. Rats were anesthetized with ketamine (80 mg/kg) and xylazine (10 mg/kg) before a laminectomy of the T10 vertebrae was performed. Spinal cord injury was performed using the Infinite Horizon device (Scheff, S. W., Rabchevsky, A. G., Fugaccia, I., Main, J. A., and Lumpp, J. E. Jr. (2003) Experimental modeling of spinal cord injury: characterization of a force-defined injury device. J. Neurotrauma 20, 179-193, which is incorporated by reference as if fully set forth), which creates a reliable contusion injury to the exposed spinal cord by rapidly applying a force-defined impact with a stainless steel-tipped impounder. Care was taken to perform laminectomies that were slightly larger than the 2.5-mm impactor tip. The vertebral column was stabilized by clamping the rostral T9 and caudal T11 vertebral bodies with forceps. The vertebral column and exposed spinal cord were carefully aligned in a level horizontal plane. During impact, the stepping motor drove the coupled rack toward the exposed spinal cord inflicting the contusion injury. The force applied to spinal cord was 200 kdyn, which produced a moderately severe injury. The impactor device was connected to a PC that recorded the impounder velocity, actual force, and displacement of the spinal cord.

At different time points following surgery (1, 3, 6, 24, 48, 72 h, and 1 week), animals in a first set (six rats per time point) were killed by sodium pentobarbital overdose (150 mg/kg). A 20-mm segment of spinal cord containing the impact epicenter was removed rapidly by laminectomy. The harvested tissue was dissected on a chilled stage and immediately transferred to a centrifuge tube containing 800 μL Triton lysis buffer [20 mmol/L Tris-HCl, 150 mmol/L NaCl, 1% Triton X-100, 5 mmol/L EGTA, 10 mmol/L EDTA, 20 mmol/L HEPES, 10% solution of glycerol, and protease inhibitor cocktail (Roche Inc., Nutley, N.J., USA)] and then briefly sonicated. Following dismembranation, the spinal cord tissue samples were centrifuged at 15,000 rpm for 1 h at 4° C., the supernatant was collected, protein levels were determined using the Protein Assay Kit (Pierce Biotechnology, Inc., Rockford, Ill., USA), the samples were then normalized to 1 μg/μL and stored at −80° C. until assay. Oxidative damage was assessed by slot immunoblotting. A 2-μg protein sample was loaded on slot-blot apparatus for optimal antibody-binding sensitivity. For lipid peroxidation, rabbit polyclonal anti-HNE antibody was applied (1:5000; Alpha Diagnostics International, Inc., San Antonio, Tex., USA). For peroxynitrite-generated 3-nitrotyrosine, rabbit polyclonal anti-nitrotyrosine antibody was employed (1:2000; Upstate USA, Inc., Charlottesville, Va., USA). To detect protein oxidation, the oxy-blot technique was used (Oxy-Blot Protein Oxidation Detection Kit; Chemicon International, Temecula, Calif., USA). The slot-blot analyses were analyzed using the Li-Cor Odyssey Infrared Imaging System (LI-COR Biosciences, Lincoln, Nebr., USA), which employs IRDye800-conjugated goat-anti-rabbit IgG (1:5000; Rockland, Gilbertsville, Pa., USA) as the secondary antibody. Preliminary studies were conducted to determine the linear range of the densitometry curve for each of the oxidative markers and, thus, verify that the densitometric readings obtained were not beyond the range of accurate quantification.

Immunohistochemistry for 3-nitrotyrosine and 4-hydroxynonenal: According to Xiong et al., at different time points following surgery (1, 3, 6, and 24 h), animals in a second set were overdosed with sodium pentobarbital (150 mg/kg) and perfused with 150 mL of 0.1 mol/L phosphate buffered saline (PBS) followed by 200 mL of 4% paraformaldehyde in PBS (pH=7.4). For cross-sections, a 5-mm spinal cord segment, centered on the injury epicenter, was dissected at different time points. For longitudinal sections, a 15-mm spinal cord segment including the impact site was dissected 24 h after injury. After harvesting, the spinal cords were immersed in 4% paraformaldehyde in PBS for 4 h. The tissues were then transferred to PBS overnight and cryopreserved in phosphate-buffered 20% sucrose for 2 days. Spinal cords were sectioned at 20 μm in a transverse or longitudinal plane, and every fifth section was transferred directly onto Superfrost plus slides (Fisher Scientific International Inc., Hampton, N.H., USA). After collecting all the spinal cord sections, the slides were placed on a tray and stored at 4° C. to dehydrate overnight after which they were stored at −20° C. until staining. On the day of staining, the frozen slides were removed from −20° C. and thawed at 20° C. for 30 min. After rinsing in 0.2 mol/L of PBS, the sections were incubated in 3% hydrogen peroxide in 0.2 mol/L of PBS for 30 min, followed by incubation in blocking buffer (5% goat serum, 0.25% Triton-X, 1% dry milk in 0.2 mol/L PBS) for 1 h, followed by the exposure to either the rabbit polyclonal anti-4-hydroxynonenal (1:5000) or anti-3-nitrotyrosine antibody (1:2000) overnight. The following day, sections were incubated for 2 h at 20° C. with biotinylated goat-anti-rabbit secondary antibody (1:200, Vector ABC-AP Kit; Vector Labs, Burlingame, Calif., USA). After rinsing, the sections were incubated in VECTASTAIN ABC reagent (avidin DH plus biotinylated horseradish peroxidase, Vector Labs) for 1 h followed by development of the staining using the Vector blue method (Vector Blue Alkaline Phosphatase Substrate Kit; Vector Labs) in the dark for 10-30 min. After reaction, spinal cord sections were counterstained with nuclear fast red (Vector Labs), dehydrated and then photographed on an Olympus Provis A70 microscope with an Olympus Magnafire digital camera (Olympus America, Inc., Melville, N.Y., USA).

Western blotting for α-spectrin breakdown products: Xiong et al. stated that fifteen micrograms of each sample were run on sodium dodecyl sulfate-polyacrylamide gel electrophoresis [3-8% (w/v) acrylamide, Bio-Rad Criterion XT precast gel] with a Tris-acetate running buffer system and then transferred to nitrocellulose membranes using a semi-dry electro-transferring unit (Bio-Rad Laboratories, Hercules, Calif., USA) at 20 mA for 15 min. The blots were probed with mouse monoclonal anti-a-spectrin antibody (1:5000, Affiniti, Inc., Ft. Lauderdale, Fla., USA; now part of Biomol International, LP Plymouth, Pa., USA), which recognizes an epitope that is common to the 280 kDa parent α-spectrin as well as each of the 150- and 145-kDa proteolytic fragments. Exposure to the primary antibody was followed by application of the secondary IRDye800-conjugated goat-anti-mouse IgG (1:5000, Rockland) for 1 h in the dark. Imaging analysis of western blots was performed using the Li-Cor Odyssey Infrared Imaging System, to quantify the content of the 145 and 150 kDa α-spectrin breakdown products (SBDP 145 and SBDP150). Each western blot included a standardized protein loading control to allow for correction in regard to intensity differences from blot to blot. This quantitative method has been employed in other studies (Kupina, N. C., Nath R., Bernath E. E., Inoue J., Mitsuyoshi A., Yuen, P. W., Wang, K. K., and Hall E. D. (2002) Neuroimmunophilin ligand V-10,367 is neuroprotective after 24-hour delayed administration in a mouse model of diffuse traumatic brain injury. J. Cereb. Blood Flow Metab. 22, 1212-1221; Hall E. D., Sullivan, P. G., Gibson, T. R., Pavel, K. M., Thompson, B. M., and Scheff, S. W. (2005) Spatial and temporal characteristics of neurodegenerations after controlled cortical impact in mice: more than a focal brain injury. J. Neruotrauma 22, 252-265, both of which are incorporated herein as if fully set forth).

Statistical analysis: Xiong et al. utilized quantitative densitometry analysis for reading the slot-blot and western immunoblot analyses. Statistical analysis was performed using the STATVIEW software package (JMP Software, Cary, N.C., USA). All values were expressed as mean±SEM. A two-way analysis of variance was first performed. If the analysis of variance revealed a significant (p<0.05) effect, post hoc testing was carried out to compare individual post-traumatic time points to the sham, non-injured group by Fisher's protected least significant difference (PLSD) test. In all cases, a p<0.05 was considered significant.

Using the tests outlined in Xiong et al., and described above, any therapeutic agent, matrix, or drug delivery system can be tested. The therapeutic agent, matrix, or drug delivery system can be implanted surgically or through injection following SCI and then subsequent marker tests, such as those in Xiong et al., may be performed. Also, markers for immune response (e.g., Glial Fibrilary Acidic Protein (GFAP)) can be monitored to track inflammatory responses. Overall extent of lesion may be assessed hematoxylin or eosin staining to monitor the affect of treatment.

The matrix, as described herein, may be used alone, seeded with cells, in combination with drugs, or blended with other polymers for optimized functionality. Functions that can be optimized include degradation rate, mechanical properties, and small-scale features. Optimization includes the formulation of particles, hydrogel, or particles and hydrogel as at least a portion of an injectable scaffold that may serve as a prosthetic or site for tissue engineering. The hydrogel and/or particles may carry cells, drugs, or other polymers useful for tissue engineering. The particle and/or hydrogel may contain peptide sequences to promote cell adhesion (e.g., RGB or IKVAV), which may be incorporated in the polymer network by crosslinks, as part of the polymer monomers, or physically constrained within the network. U.S. Pat. Nos. 5,759,830; 5,770,417; 5,770,193; 5,514,378; 6,689,608; 6,281,015; 6,095,148; 6,309,635; and 5,654,381 relate to synthesis of polymers, optimizing polymers, seeding polymers with cells, and preparing tissue scaffolds and are incorporated by reference herein in their entirety as if fully set forth.

Although treatment of spinal cord injury represents a preferred embodiment, the drug delivery system may be utilized to treat peripheral nerve injury, stroke, myocardial infarction, chronic heart failure, diabetes, circulatory shock, chronic inflammatory diseases, cancer, and neurodegenerative disorders. Additional maladies that the drug delivery system can be adapted to treat include, but are not limited, to those described in Pacher, P., Beckman, J. S., Liaudet, L. (2007) Nitric oxide and peroxynitrite in health and disease. Physiol. Rev. 87, 315-424, which is incorporated by reference as if fully set forth. To treat any of these conditions, including spinal cord injury, the drug delivery system is administered at the site of injury caused by the malady. Administration can be by any means, which includes but is not limited to surgical implantation or injection.

The skilled artisan will appreciate that two or more of the embodiments described above may be compatible with one another and may be implemented in combination with one another.

In further alternate embodiments, free radical scavengers, for example, ascorbic acid, may be incorporated in a matrix as described above and the combination may be utilized as a preservative in the food and packaging industries. For example, a an edible matrix can be provide with an edible antioxidant, preferably a poly lactic acid based polymer matrix is provided with vitamin C.

### EXAMPLES

### Example 1

### Multiblock Copolymer Synthesis—PGA-PEG-PGA (poly(glycolide)-b-poly(ethylene glycol)-b-poly(glycolide))

This polymer is an example of a temperature-sensitive block copolymer consisting of hydrophobic end groups polymerized on either side of a hydrophilic polymer. It is an amphiphilic triblock copolymer for a temperature-sensitive hydrogel. In this case ring opening polymerization is used to construct hydrophobic end chains.

Materials

1 gram poly(ethylene-glycol), MW 4000=0.00025 mol;

0.05 mol glycolide=5.805 g glycolide; and

Stannous octanoate as a catalyst, 0.025 wt. %=1.7 mg stannous octanoate, density=1.251 g/mL, 1.36 microliters.

Method

1. Dry PEG and glycolide were placed in an oven dried schlenk flask under vacuum for 40 minutes with stirring via a stir bar.

2. The PEG and glycolide were melted at 150° C. and a 15 microliter droplet of the catalyst in acetone was added.

3. The reaction was allowed to proceed until the melt became amber and viscous.

### Example 2

### Multiblock Copolymer Synthesis—PLGA-PEG-PLGA (poly(lactide-co-glycolide)-b-poly(ethylene glycol)-b-poly(lactide-co-glycolide))

This is another example of an amphiphilic triblock copolymer for a temperature-sensitive hydrogel.

Materials

PEG-4000, 0.000125 mol, 0.5 g;

Glycolide, 0.00625 mol, 0.725625 g;

D,L-Lactide, 0.00625 mol, 0.9008125 g; and

Stannous Octanoate as a catalyst, 0.05% of total feed=0.85 mg Stannous Octanoate, Density=1.251 g/mL, 0.68 microliters.

Method

1. PEG, glycolide and lactide were charged to the flask. The flask was placed under vacuum and then filled with argon.

2. The PEG, glycolide and lactide were melted at 150° C. and add a 15 microliter droplet of the catalyst in acetone was added

3. The reaction was allowed to proceed for 1 hour and 45 minutes

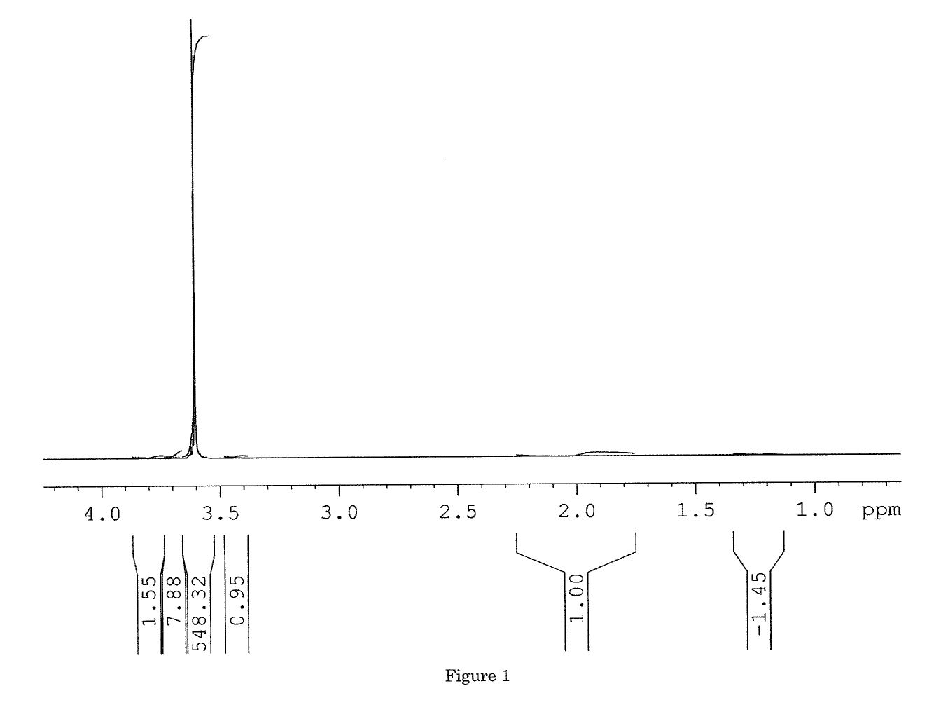

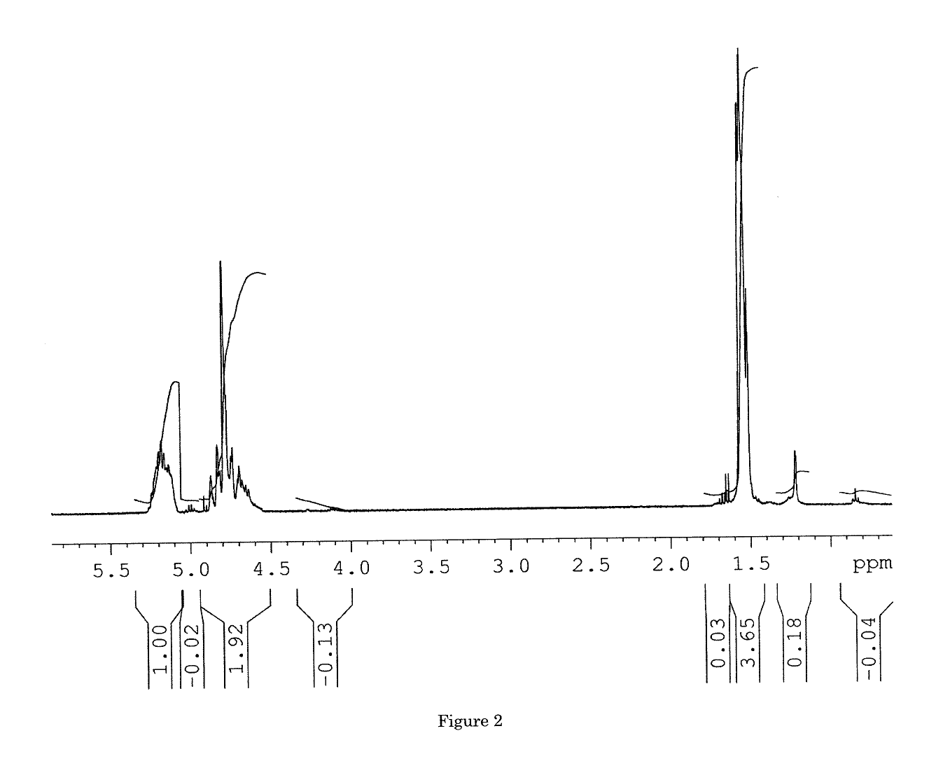

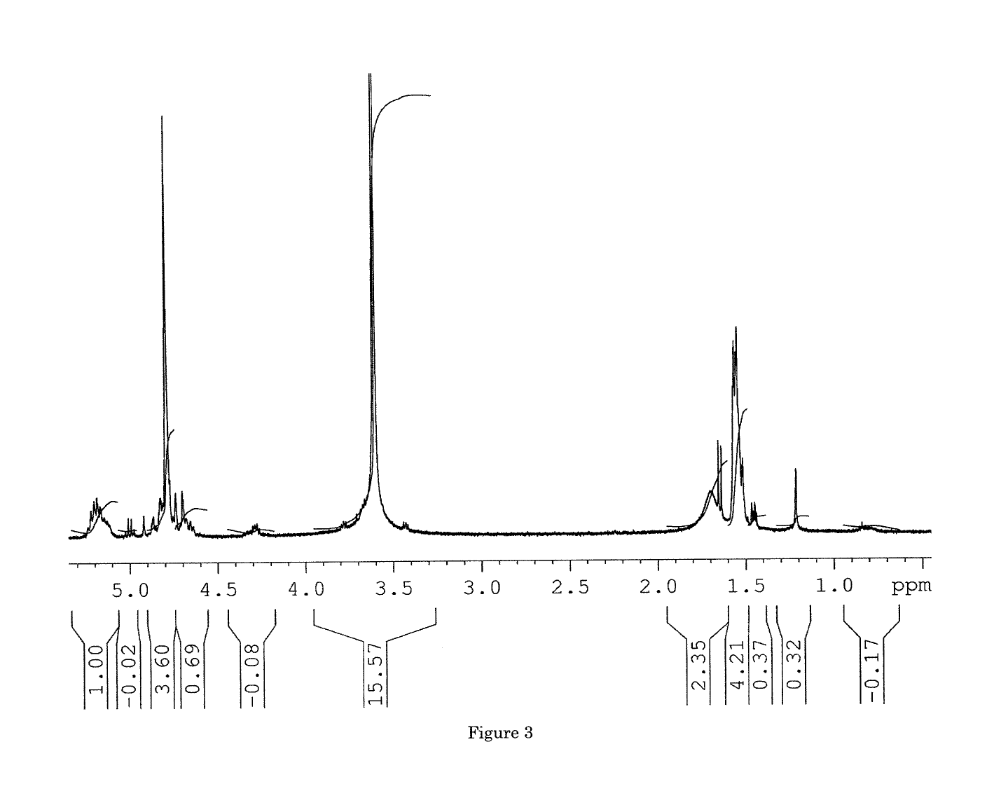

FIGS. 1, 2 and 3 demonstrate successful synthesis of CP-PLGA-pPEG-PLGA-1 triblock copolymer. The methylene of PEG shows as a shift at 3.5-3.7 ppm (4 Hydrogens), the methylene of PGA shows as a shift at 4.6-4.9 ppm (2 Hydrogens), and the methine of PLA shows as a shift at 5.2 ppm (1 Hydrogen).

These peaks had the following areas: PEG=15.57/4=3.8925; PGA=3.6/2=1.8; and PGA=1. PEG (Fluka) has a polymer molecular weight=4000 g/mol, where the monomer MW=44, and the degree of polymerization=91. The PLA monomer has a MW=72, a degree of polymerization=91/3.8925=23.38, and a polymer MW=1683.24. The PGA monomer MW=58, the degree of polymerization=91/(3.8925/1.8)=42.08, and the polymer has a MW=2440.69. The total PLGA-PEG-PLGA Molecular Weight: 8881.38 g/mol. The PEG block has a molecular weight of approximately 4000 g/mol. Each PLGA block is around 4881.38 g/mol, with a PLGA ratio of lactic acid to glycolic acid monomers of approximately 36:64.

### Example 3

### Multiblock Copolymer Synthesis—CTA-CP-PLGA-pPEG-PLGA-CTA

This polymer serves as an example of a macro-chain-transfer-agent for reversible addition-fragmentation chain transfer (RAFT) polymerization of a multiblock copolymer. In this case, the macro-chain-transfer-agent is a tri-block and can be used to make amphiphilic multiblock copolymers with 5 or more blocks.

Materials

CP-PLGA-pPEG-PLGA as previously described, or any other polymer with hydroxyl end group difunctionality;

S-(thiobenzoyl)-thioglycolic acid (CTA) or another acid chain transfer agent; and

Dicyclohexyl carbodiimide (DCC), to activate the chain transfer agent.

Methods

1. 100 mg CP-PLGA-pPEG-PLGA-1 (1.126×10⁻⁵ mol at 8881.38 g/mol) was dissolved in 1 mL anhydrous dichloromethane.

2. 2× mol of DCC compared to CP-PLGA-pPEG-PLGA-1 (4.65 mg) and 5× mol of CTA compared to CP-PLGA-pPEG-PLGA-1 (11.95 mg) were added to a round bottom flask with a stirrer.

3. The flask was put under vacuum for 1 hour.

4. The vacuum was replaced with argon.

5. 1 mL of anhydrous dichloromethane was added to the flask.

6. The dissolved polymer was added dropwise to the flask and stirred (300 rpm) at room temperature overnight.

7. The resulting solution was precipitated in 100 mL ethyl ether.

8. The mixture was filtered by vacuum filter through filter paper and the precipitate was dried.

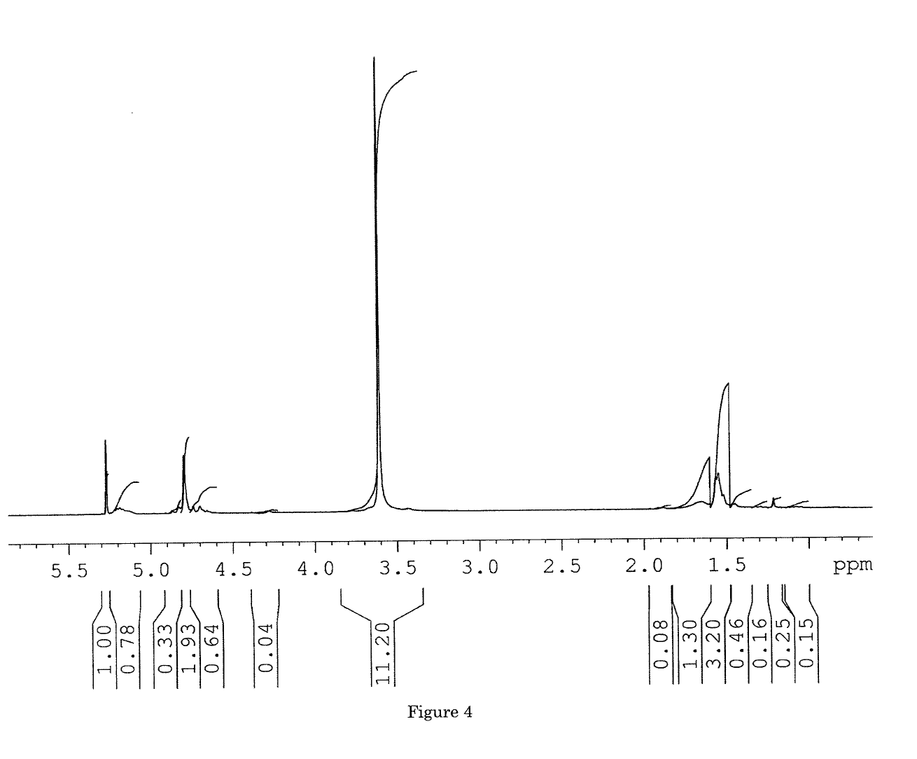

Referring to FIG. 4, the presence of the chain transfer agent by ¹H-NMR analysis is not evident. However a refined process, Example 4, was developed to increase the efficacy of coupling a chain transfer agent to a polymer with hydroxyl end groups.

### Example 4

### Multiblock Copolymer Synthesis—DJS-CP-CTA-Cl

An acid chloride form of a RAFT chain transfer agent was developed to increase reactivity with polymers with hydroxyl end groups. This can be useful to facilitate the coupling of the acid chain transfer agent (CTA) as previously described when it is undesirable to use a base catalyst, such as 4-dimethylaminopyridine (DMAP), due to the risk of increasing base-catalyzed ester hydrolysis of alpha-hydroxy-acid polymer blocks or other possible ester blocks in the macromer.

Materials

S-(thiobenzoyl)-thioglycolic acid or other CTA

Oxalyl Chloride

Method

1. 0.5 g S-(thiobenzoyl)-thioglycolic acid was dissolved in anhydrous dichloromethane in a 50 mL dried round bottom flask with a stirrer and cooled to 0° C. by immersing in an ice-water bath.

2. 1.2 mol equivalent of oxalyl chloride was added slowly under nitrogen and the solution was allowed to reach room temperature, stirring for three hours

3. The solution was concentrated under reduced pressure to yield acid chloride, or left in dichloromethane

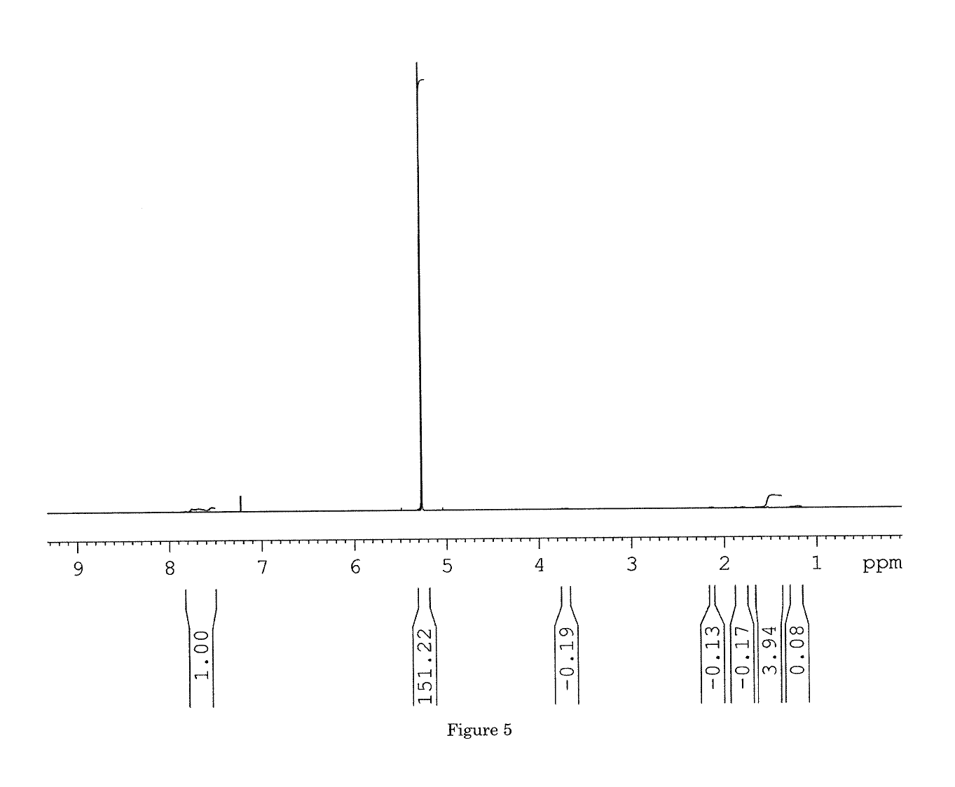

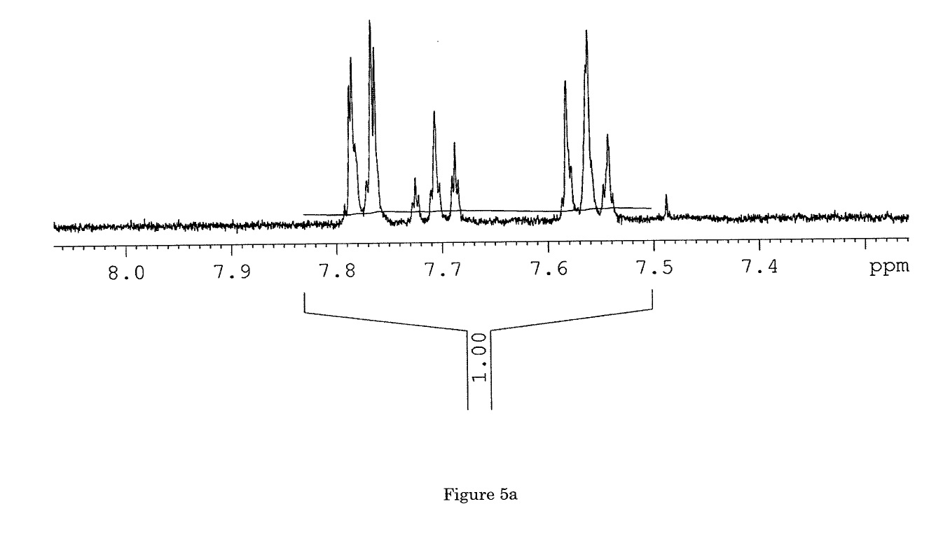

FIG. 5 illustrates the ¹H-NMR spectrum of the resulting S-(thiobenzoyl) thioglycolic acid chloride DJS-CP-thiobenzoyl-thioglycolic acid chloride-1.

### Example 5

### Multiblock Copolymer Synthesis—Coupling of CTA-Cl to CP-PLGA-pPEG-PLGA-1

This method demonstrates the success of using an acid chloride form of a chain transfer agent for coupling to a polymer to create a macro chain transfer agent for RAFT polymerization of blocks contributing to a thermo-sensitive copolymer.

Method

1. 100 mg dry CP-PLGA-pPEG-PLGA-1 in 1 mL anhydrous dichloromethane was placed in a Schlenk flask and then 7.84 microliters of triethylamine were added.

2. The mixture was cooled to 0° C. under inert gas.

3. DJS-CP-thiobenzoyl-thioglycolic acid chloride-1 (0.346 mL in dichloromethane for 5× mol/mol of acid chloride compared to polymer) was slowly added.

4. The reaction was allowed to reach room temperature and react for 24 hours.

5. The solution was filtered to remove triethylamine salts.

6. The filtered solution was precipitated in ethyl ether to remove unreacted acid chloride and triethylamine.

7. The precipitate was vacuum dried after filtering.

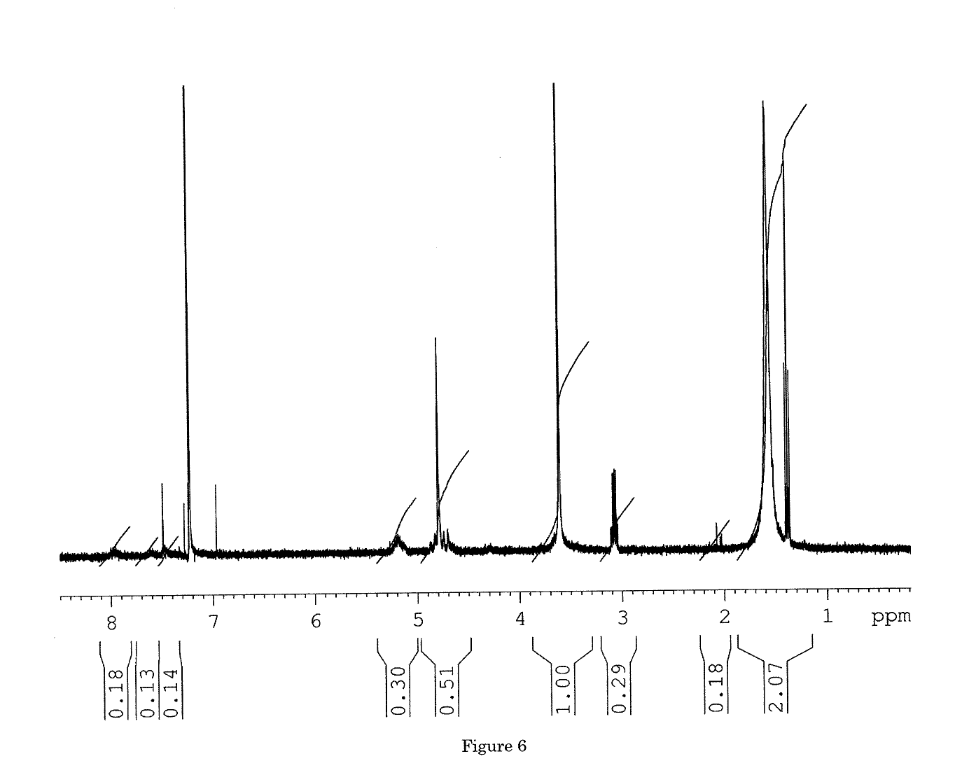

FIG. 6 shows successful functionalization of the CP-PLGA-pPEG-PLGA-1 copolymer with RAFT chain transfer agent end groups (7.6, 8.0 ppm) to create a macro chain transfer agent for RAFT polymerization to add further polymer blocks. The RAFT polymerization process can be used to add oligo ethylene glycol methyl methacrylate to the tri-block to create a temperature-sensitive a biocompatible biodegradable pentablock copolymer for an injectable hydrogel drug delivery device or injectable tissue engineering scaffold.

### Example 6

### Multiblock Copolymer Synthesis—PGS-CTA

Hydroxyl groups of Poly(glycerol-co-sebacic) acid can be functionalized with a RAFT chain transfer agent as previously described for CP-PLGA-pPEG-PLGA-1 using either the acid or acid chloride form of the chain transfer agent. For example, hydroxyl groups of poly(glycerol-co-sebacic) acid can be functionalized via RAFT with oligo (ethylene glycol methyl methacrylate) to create a graft copolymer that can form a temperature sensitive elastomeric network.

Materials 1

Poly(glycerol-co-sebacic acid);

S-(Thiobenzoyl)-thioglycolic acid chain transfer agent (CTA) or another acid CTA;

Dicyclohexyl carbodiimide (DCC), to activate the CTA; and

DMAP, as a base catalyst.

Method 1

1. 0.5 g PGS (˜1.95 mmol hydroxyl groups) were dissolved in 5 mL anhydrous dichloromethane.

2. Equimolar DCC (0.402 g) compared to hydroxyl groups in PGS and excess CTA (0.414 g) compared to PGS were added to the round bottom flask with a stirrer. Then 0.1 mol of DMAP compared to hydroxyl groups in PGS was added

3. The flask was placed under vacuum for 1 hour.

4. The vacuum was replaced with argon.

5. Anhydrous dichloromethane was added to the flask to dissolve DCC, CTA and DMAP

6. Dissolved PGS polymer from step 1 was added dropwise to the flask and stirred at room temperature (300 rpm) overnight

7. The resulting solution was precipitated in 250 mL ethyl ether

8. The products were vacuum filtered through filter paper and the precipitate was dried.

Materials 2

Poly(glycerol-co-sebacic acid)

DJS-CP thiobenzoyl thioglycolic acid chloride-1 or another acid chloride chain transfer agent; and

Triethylamine, as a base catalyst

Method 2

1. 100 mg dry PGS in 1 mL anhydrous dichloromethane was placed in a Schlenk flask. Then, triethylamine (equimolar to acid chloride) was added.

2. The mixture was cooled to 0° C. under inert gas.

3. DJS-CP-thiobenzoyl-thioglycolic acid chloride-1 (2× mol/mol of acid chloride compared to desired functionalization of polymer hydroxyl groups) was slowly added.

4. The reaction was allowed to reach room temperature and react for 24 hours.

5. The solution was filtered to remove triethylamine salts.

6. The filtered solution was precipitated in ethyl ether to remove unreacted acid chloride and triethylamine.

7. The precipitate was filtered and vacuum dried.

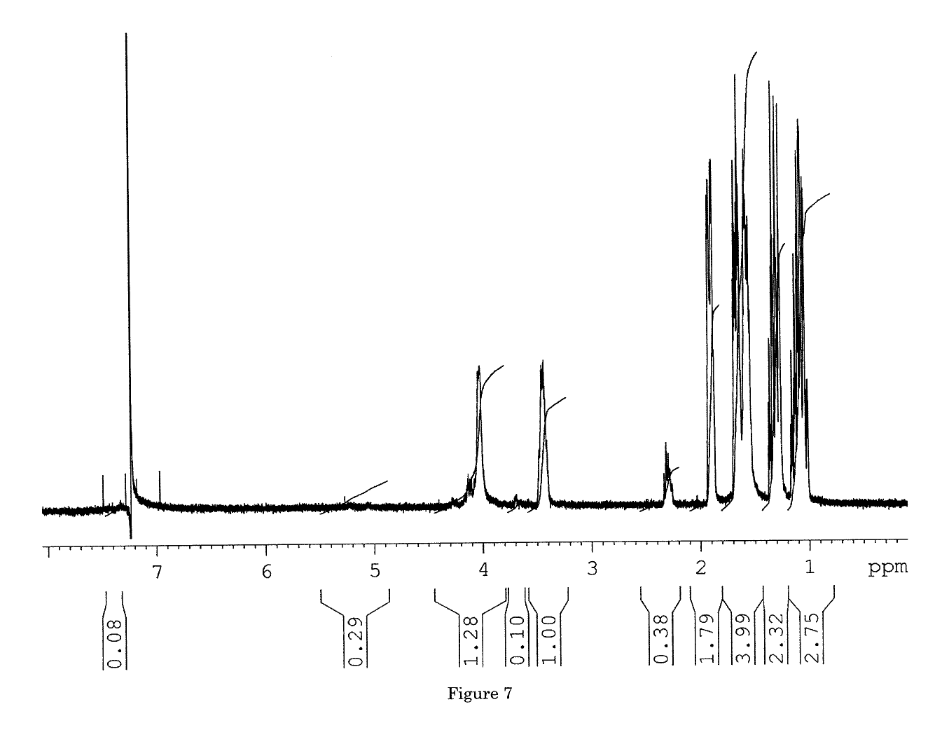

As shown in FIG. 7, the process yielded CP-PGS-CTA poly(glycerol-co-sebacic acid) functionalized with a S-thiobenzoyl-thioglycolic acid chain transfer agent.

### Example 7

### Injectable Hydrogel Tested In Vitro and In Vivo

Injectable Hydrogel Tested In Vitro

Water soluble polymer compounds were identified that gel rapidly under physiologic conditions and also exhibit tunable swelling properties. These polymer compounds included the following:

1. Ethoxylated trimethylolpropane tri-3-mercaptopropionate (ETTMP1300). CAS 345352-19-4. MW 1300 g/mol. Bruno Bock GmbH, Marschacht, Germany.

2. Ethoxylated trimethylolpropane tri-3-mercaptopropionate (ETTMP700). CAS 345352-19-4. MW 700 g/mol, Bruno Bock GmbH, Marschacht, Germany.

3. Poly(ethylene glycol)diacrylate (PEGDA400). CAS 26570-48-9. MW 400 g/mol. Polysciences, Warrington, Pa., USA.

4. Poly(ethylene glycol)diacrylate (PEGDA4000). CAS 26570-48-9. MW 4000 g/mol. Polysciences, Warrington, Pa., USA.

Formulation

ETTMP1300 and ETTMP700 contain three thiol functional groups. PEGDA400 and PEGDA4000 contain two acrylate functional groups. The compounds were combined so that the ratio of thiol and acrylate functional groups were equimolar. The compounds were then dissolved in phosphate buffered saline (PBS) pH 7.4 (Gibco, Carlsbad, Calif.) at 20, 25, 30 w/v % total polymer in solution. Any combination of acrylate and thiol containing polymers may be used. But higher molecular weight acrylates led to greater swelling. To decrease swelling, lower molecular weight acrylates (e.g., having a molecular weight similar to that of PEGDA400) may be utilized.

Conversions Rates from Sol to Gel

200 microliters of polymer and saline solutions (ETTMP1300/PEGDA400) of different concentrations were placed in 1.5 mL Eppendorf tubes and incubated at 37° C. or left at room temperature at 22° C. in absence of light. Table 1 shows conversion times from sol to gel, which was considered to have occurred when the solution no longer flowed, monitored by turning tubes upside-down and agitating manually (3 samples per group).

TABLE 1
Conversion rates as a function of thiol-acrylate
concentration in phosphate buffered saline pH 7.4

Temperature

| Concentration (w/v %) | 22° C. | 37° C. |
|---|---|---|
| 20 | Did not gel after 11 hours | 25 mins |
| 25 | 25 mins | 15 mins |
| 30 | 15 mins | 10 mins |

Swelling Tests

1.5 mL gels (n=6) were cured at 37° C., mass and volume determined, and subsequently put into 300 mL phosphate buffered saline pH 7.4 at 37° C. and allowed to equilibrate for 7 days.

A swelling ratio can be defined as the ratio of the volume of the hydrogel at equilibrium to the initial volume of the hydrogel after curing. Another informative measure is the ratio of initial hydrogel polymer weight percent to the equilibrium hydrogel polymer weight percent. Initial polymer weight percent is determined by formulation. Equilibrium polymer weight percent is determined by measuring the hydrogel wet mass following equilibration. Subsequently, the hydrogel is freeze dried and the dry mass is measured. The equilibrium polymer weight percent is given by the ratio of dry mass to wet mass. Comparing this to the initial polymer weight percent also gives an indication of swelling. The extent of conversion and degradation of the hydrogel can be monitored by comparing the dry mass following equilibration to the initial polymer mass added to the hydrogel.

Injectable Hydrogel Tested In Vivo

A 25 w/v % solution of ETTMP1300 and PEGDA400 was made by mixing from two stock solutions: Vial 1, 1720 mg ETTMP1300 in 3.28 mL PBS; and Vial 2, 794 mg PEGDA400 in 4.21 mL PBS. Stock solutions were sterile filtered (0.2 μm Supor membrane Acrodisc syringe filter, PALL life sciences), and pipetted into separate 200 μL aliquots under sterile conditions.

Contusion injuries were performed on 250 g rats under anesthesia (isoflurane) using an Infinite Horizon impactor (250 kdynes) following exposure of the spinal cord at T8 by laminectomy.

At 6 hours (4 rats) or 3 days (4 rats) post-injury, rats were re-anesthetized and the spinal cord re-exposed at the size of contusion. Using a stereotaxic frame, a 25 μL syringe (Hamilton 1802RN, 26 gauge blunt needle) loaded with 5 μL saline and 15 μL of the thiol-acrylate gel solution (by mixing of two 200 μL aliquots containing ETTMP1300 and PEGDA400 in PBS), was inserted 1.1 mm into the spinal cord to the epicenter of the injury (measured from the dorsal medial surface of the dura). Gel was injected at a rate of 3 μL/min over 5 minutes. The additional saline in the syringe served to prevent adhesion of the gel to the syringe, so that upon removal of the syringe the gel was not removed. The leftover hydrogel following mixing of the aliquots cured at room temperature in approximately 20 minutes. Inside the spinal cord, the gel is was assumed to have cured completely in less than 7 minutes. Upon removal of the syringe, small amounts of residual gel was observed when the injection took between 5 and 7 minutes.

Rats will be monitored for function over a period of 14 days using Basso Beattie Bresnahan (BBB) scoring alongside controls receiving injuries but no injections. After two weeks, rats will be euthanized and spinal cords collected for tissue analysis to assess the size and characteristics of injury (hematoxylin and eosin staining) as well as inflammatory markers (GFAP, Ibal immunohistochemistry).

### Example 8

### Methylprednisolone Microparticles

Fabrication

Instructions for preparing a single batch of single emulsion microparticles (˜250 mg) are as follows.

Solution Preparation

As a simple precaution against contamination, wash all beakers with ethanol and acetone. Make an 800 mL aqueous solution using distilled deionized water containing 0.25 wt. % poly (vinyl alcohol) (PVA) and 0.5 M sodium chloride (NaCl). Dissolve the solids using a hot plate to speed the process, and allow the solution to reach room temperature (or the homogenization may cause foaming). Make a 1 liter 0.5 M sodium chloride solution. Weigh 450 mg poly(lactide-co-glycolide) (PLGA) (ex. Boehringer Ingelheim RG502H, MW 11,600 g/mol) and dissolve in 1.1 mL methylene chloride (DCM). Weigh 50 mg methylprednisolone sodium succinate (MPss) and dissolve in 400 μl methanol. Combine methylene chloride and methanol solutions.

Homogenization

Prepare homogenizer with a middle-sized head by cleaning with water, acetone, and then water again. Lower homogenizer head into 800 mL PVA/NaCl solution and set speed to 6500 rpm. Inject combined PLGA/DCM/MPss/methanol mixture with a glass pipette and homogenize for 20 seconds. Pull head up and rinse off with water. Pour homogenized solutions (approx. 800 mL) into 1 liter 0.5M NaCl solution. Stir for 1 hour at 400 rpm on a stir plate with a magnetic stirrer.

Filtration, Washing, and Lyophilization

Filter stirred 1.8 liter solution to remove PVA and DCM under vacuum through and ethyl acetate filter. Rinse and collect microparticles from filter with distilled water. Pour suspended microparticles into 50 mL Falcon tubes. Centrifuge tubes at 1500 rcf for 3 minutes. Replace supernatant with distilled deionized water and resuspend microparticles. Repeat three times. After the final centrifugation step, remove supernatant and resuspend in 5 mL distilled deionized water. Lyophilize by freezing the suspension in the Falcon tubes with liquid nitrogen and placing under mTorr vacuum in a lyophilizer. Aliquot dry microparticles into Eppendorf tubes and package for electron beam sterilization (3 mRad).

Release of Methylprednisolone from Microparticles Suspended in Thiol-Acrylate Hydrogels In Vitro

Release Study Setup

Microparticles were suspended in hydrogels by vortexing 16 mg of methylprednisolone PLGA microparticles (fabricated as described above) with 160 μL of 25 w/v % of thiol-acrylate hydrogel solution. 50 μL of the suspension was pipetted into a 15 mL falcon tube three times, and gels were cured at the bottom of the tubes. 10 mL PBS was added to the tubes, on top of the hydrogels, and sealed tubes were placed on top of an orbital shaker at 37° C. At regular time intervals over 14 days, 300 μL aliquots were collected from the supernatant.

Analysis of Drug Release by HPLC

Release rates of methylprednisolone from the hydrogel-microparticle depots were measured by analyzing samples taken at various time points by high pressure liquid chromatography (HPLC).

An Agilent 1100 HPLC system was used, with a LW detector at 238 nm. An Atlantis dC18 5 μm 4.6 mm×250 mm column (Waters, Ireland) was used. The mobile phase contained acetonitrile, water and formic acid (60:40:1 ratio by volume), at a flow rate of 1 mL/min. The injection volume was 5 μL. Methylprednisolone sodium succinate had a retention time under these conditions of 8.4 minutes. A standard curve based on peak areas was generated using 6 samples diluted geometrically from 85 to 2.66 μg/mL with a linear fit with an r-squared value of 0.9997.

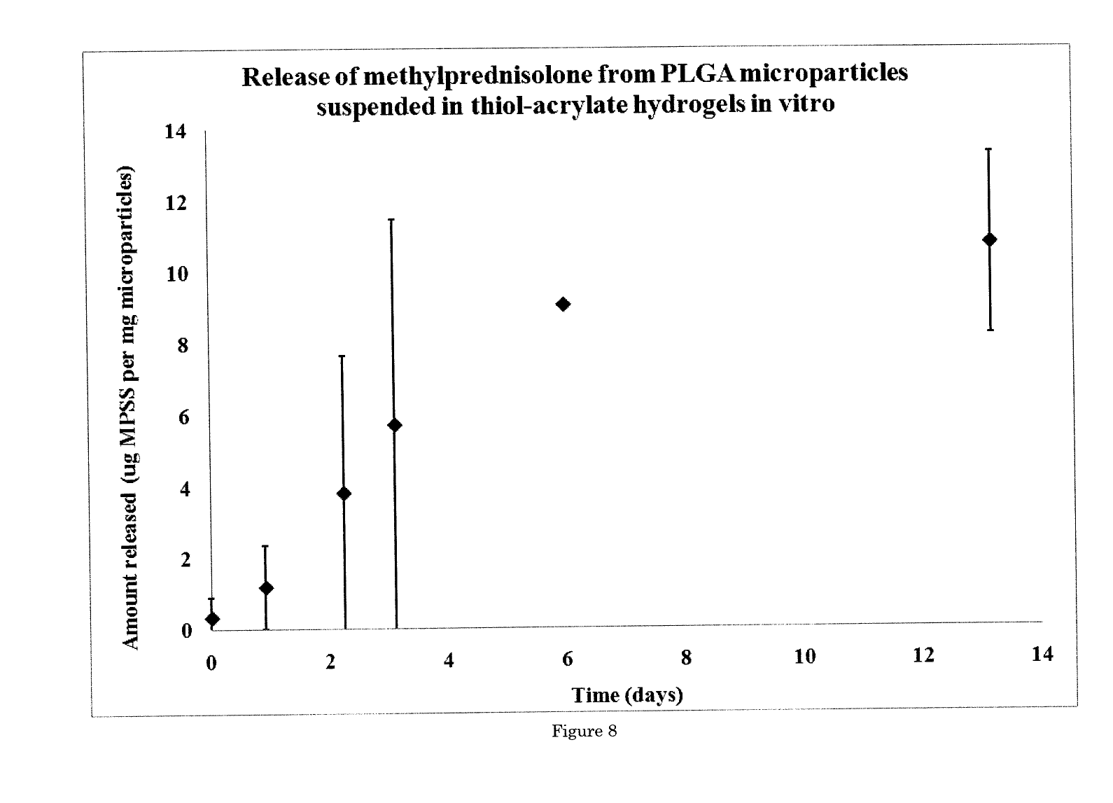

A release curve based on three hydrogel-microparticle with 5 mg microparticles in 50 μL hydrogel is shown in FIG. 8.

### Example 9

### Dosage for the Treatment of Traumatic Spinal Cord Injury

In a rat spinal cord, it was feasible to inject 15 μL of hydrogel into the intradural intramedullary epicenter of a contusion injury. Based on the formulation from example 8 (25 w/v % of thiol-acrylate hydrogel solution plus 1.5 mg methylprednisolone containing microparticles), this corresponds to a dosage of 15 μg of methylprednisolone sodium succinate released in a controlled manner over 1-2 weeks. In a clinical setting, based on the fact the diameter of the human spinal cord is approximately 10 mm in diameter at T8 versus 2.8 mm in the rat, it may be feasible to inject 150 μL of the hydrogel into a human spinal cord injury with the same impact. This would correspond to a dose of 150 μg of methylprednisolone sodium succinate released directly at the injury site during the time course of secondary injury.

### Example 10

### Peripheral Nerve Treatments

In the case of inflammation due to injury to peripheral nerves caused by trauma or chronic degeneration (e.g., nerve root impingement), it may be feasible to inject the hydrogel close to the site of injury. In these cases, the dosage may vary depending on the available space surrounding the site of inflammation. For example, if 1 mL of the hydrogel can be injected adjacent to the nerve, a dose of 1 mg methylprednisolone sodium succinate, released over 1-2 weeks may be administered.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A method of treating an injury, which is capable of causing secondary injury having a time course, at a site of the injury in a patient comprising administering a drug delivery system having a matrix including one or more polymers and one or more therapeutic agents to the patient at the site of injury;
the polymers having a molecular weight less than or equal to 4000 g/mol, at least one type of monomer selected from the group consisting of ethylene glycol monomers and ethylene oxide monomers and functional groups selected from the group consisting of thiol functional groups, acrylate functional groups, methacrylate functional groups and vinyl functional groups, and
the one or more therapeutic agents including one or more type of steroid selected from the group consisting of methylprednisolone, dexamethasone, prodrugs of methylprednisolone, prodrugs of dexamethasone, pharmaceutically acceptable salts of methylprednisolone, pharmaceutically acceptable salts of dexamethasone, pharmaceutically acceptable salts of prodrugs of methylprednisolone, and pharmaceutically acceptable salts of prodrugs of dexamethasone, and
wherein the matrix releases the steroid at the site of injury over the time course after the step of administering.

2. The method of claim 1, wherein the step of administering includes injecting the drug delivery system into the patient at the site of the injury.

3. The method of claim 2, wherein at least a portion of the polymers are a temperature-sensitive hydrogel.

4. The method of claim 3, wherein a plurality of the monomers are the monomers including the thiol functional groups and monomers including the acrylate functional groups, and the temperature-sensitive hydrogel includes thiol esters of at least one of the thiol functional groups and at least one of the acrylate functional groups.

5. The method of claim 3, wherein the one or more polymers are selected from the group consisting of poly(glycerol-co-sebacic acid) acrylate; multiblock copolymers of poly(lactide-co-glycolide) and poly(ethylene glycol) or oligo (ethylene glycol) methyl methacrylate; graft copolymers of poly(glycerol-co-sebacic acid) and poly(ethylene glycol), oligo (ethylene glycol) methyl methacrylate or poly(N-isopropylacrylamide); and thiol esters of ethoxylated trimethylolpropane tri-3-mercaptopropionate and poly(ethylene glycol)diacrylate.

6. The method of claim 3, wherein the one or more polymers include thiol esters of ethoxylated trimethylolpropane tri-3-mercaptopropionate and poly(ethylene glycol)diacrylate.

7. The method of claim 3, wherein the temperature-sensitive hydrogel is biodegradable and the hydrogel components are biodegradable or biocompatible and excretable, or the hydrogel includes a mixture of biodegradable components and biocompatible and excretable components.

8. The method of claim 2, wherein the matrix includes particles.

9. The method of claim 8, wherein the particles are microparticles, nanoparticles, or a combination of microparticles and nanoparticles.

10. The method of claim 8, wherein the particles include a biodegradable polymer, a biocompatible polymer that is excretable, or a biodegradable polymer that includes biocompatible and excretable components.

11. The method of claim 8, wherein the particles include a polyester.

12. The method of claim 8, wherein the particles include one or more polymer selected from the group consisting of poly(lactide-co-glycolide); polylactide, polyglycolide; and poly(carboxyphenoxy propane)-co-sebacic acid).

13. The method of claim 8, wherein the particles are microparticles including poly(lactide-co-glycolide).

14. The method of claim 2, wherein the one or more therapeutic agents further include one or more substance selected from the group consisting of inhibitors of NOS or NO production, antioxidants, spin traps, peroxynitrite scavengers, pharmaceutically acceptable salts of inhibitors of NOS or NO production, pharmaceutically acceptable salts of antioxidants, pharmaceutically acceptable salts of spin traps, and pharmaceutically acceptable salts of peroxynitrite scavengers.

15. The method of claim 2, wherein the one or more therapeutic agents further include a substance selected from the group consisting of an antioxidant or antioxidants, tempol (4-hydroxy-2,2,6,6-tetramethylpiperidine-1-oxyl), uric acid, minocycline, and MnTBAP, or pharmaceutically acceptable salts thereof.

16. The method of claim 2, wherein the one or more therapeutic agents include minocycline.

17. The method of claim 2, wherein the matrix is functionalized with the one or more therapeutic agents.

18. The method of claim 2, wherein the site of the injury is in the spinal cord and the step of injecting includes intradural intrameduallary injection.

19. The method of claim 2, wherein the site of the injury is a peripheral nerve.

20. The method of claim 2, wherein the polymers are part of a temperature-sensitive hydrogel and the matrix includes the temperature-sensitive hydrogel and particles.

21. The method of claim 20, wherein the one or more therapeutic agents are dissolved or dispersed in the temperature-sensitive hydrogel, the particles, or both the temperature-sensitive hydrogel and the particles.

22. The method of claim 21, where the one or more therapeutic agents are a plurality of therapeutic agents and one or more of the plurality of therapeutic agents is dissolved or dispersed in the hydrogel and one or more other ones of the plurality of therapeutic agents is dissolved or dispersed in the particles.

23. The method of claim 20, wherein the one or more polymers includes thiol esters of ethoxylated trimethylolpropane tri-3-mercaptopropionate and poly(ethylene glycol)diacrylate, and the particles include microparticles having poly(lactide-co-glycolide).

24. The method of claim 23, wherein the one or more therapeutic agents further include one or more substance selected from the group consisting of inhibitors of NOS or NO production, antioxidants, spin traps, peroxynitrite scavengers, pharmaceutically acceptable salts of inhibitors of NOS or NO production, pharmaceutically acceptable salts of antioxidants, pharmaceutically acceptable salts of spin traps, and pharmaceutically acceptable salts of peroxynitrite scavengers.

25. The method of claim 23, wherein the one or more therapeutic agents further include minocycline or a pharmaceutically acceptable salt thereof.

26. The method of claim 23, wherein the one or more therapeutic agents are dissolved or dispersed in the microparticle.

27. The method of claim 23, wherein the site of injury is in the spinal cord and the step of injecting includes intradural intrameduallary injection.

28. The method of claim 23, wherein the site of injury is a peripheral nerve.

29. The method of claim 23, wherein one or both of the hydrogel and microparticles are functionalized with the one or more therapeutic agent.

30. The method of claim 2, wherein the one or more therapeutic agents further include vitamin C, vitamin E, pharmaceutically acceptable salts of vitamin C, or pharmaceutically acceptable salts of vitamin E.
